(19) United States
(12) Patent Application Publication
(10) Pub. No.: US 2024/0122480 A1
(43) Pub. Date: Apr. 18, 2024

(54) SYSTEM AND APPARATUS FOR SENSING ABNORMALITY OF BREAST IMPLANT

(71) Applicant: Jae Hong Kim, Seoul (KR)

(72) Inventor: Jae Hong Kim, Seoul (KR)

(21) Appl. No.: 18/394,375

(22) Filed: Dec. 22, 2023

Related U.S. Application Data
(63) Continuation of application No. PCT/KR2022/014862, filed on Sep. 30, 2022.

(30) Foreign Application Priority Data
Oct. 3, 2021 (KR) 10-2021-0131279

(51) Int. Cl. A61B 5/00

(52) U.S. Cl. CPC A61B 5/0053; A61B 5/686

(57) ABSTRACT

An apparatus for sensing abnormality of a breast implant is proposed. The apparatus may include a sensor signal receiver to receive a pressure signal indicating a pressure applied to a breast implant inserted in a body from at least one sensor module disposed on the breast implant. The apparatus may also include a memory to store a predetermined reference pressure value, a predetermined first tolerance, and a predetermined second tolerance. The apparatus may further include an abnormality determining processor configured to determine whether there is an abnormality in the breast implant based on a pressure value indicated by the pressure signal received by the sensor signal receiver and the reference pressure value, the first tolerance, and the second tolerance stored in the memory.

### CROSS-REFERENCE TO RELATED APPLICATIONS

This application is a continuation of International Application No. PCT/KR2022/014862, filed Sep. 30, 2022, which is based upon and claims the benefit of priority from Korean Patent Application No. 10-2021-0131279 filed Oct. 3, 2021, the contents of which are incorporated herein by reference in their entirety.

### BACKGROUND

### Technical Field

The present disclosure relates to a system and an apparatus for sensing an abnormality of a breast implant.

### Description of Related Technology

A breast with beauty and volume is the shared wishes of most women. The breast implant is one of the easiest and most effective methods to realize the wishes of such women.

The breast reconstruction employing the breast implant is a method of securing a space for inserting the breast implant in a breast and increasing the volume of the breast by inserting the breast implant in the secured space.

### SUMMARY

According to some embodiments of the present disclosure, an apparatus for sensing abnormality of a breast implant includes a sensor signal receiving unit configured to receive a pressure signal indicating a pressure applied to a breast implant inserted in a body from at least one sensor module disposed on the breast implant, a storing unit configured to store a predetermined reference pressure value, a predetermined first tolerance, and a predetermined second tolerance, and an abnormality determining unit configured to determine whether there is an abnormality in the breast implant based on a pressure value indicated by the pressure signal received by the sensor signal receiving unit and the reference pressure value, the first tolerance, and the second tolerance stored in the storing unit. The sensor signal receiving unit is configured to receive a first pressure signal from the sensor module in a predetermined period or in an arbitrary manner by an operation of a user. The abnormality determining unit is configured to compare a first pressure value indicated by the first pressure signal with the reference pressure value. When an absolute value of a difference between the first pressure value and the reference pressure value is larger than the first tolerance, the sensor signal receiving unit is configured to receive a second pressure signal from the sensor module after a predetermined first time lapse. The abnormality determining unit is configured to compare a second pressure value indicated by the second pressure signal with the reference pressure value, when an absolute value of a difference between the second pressure value and the reference pressure value is larger than the first tolerance, to compare an absolute value of a difference between the first pressure value and the second pressure value with the second tolerance, and when the absolute value of the difference between the first pressure value and the second pressure value is smaller than the second tolerance, to determine the abnormality in the breast implant.

According to some embodiments of the present disclosure, an apparatus for sensing abnormality of a breast implant includes a sensor signal receiving unit configured to receive a pressure signal indicating a pressure applied to a breast implant inserted in a body from at least one sensor module disposed on the breast implant, a storing unit configured to store a predetermined first tolerance and a predetermined second tolerance, and an abnormality determining unit configured to determine whether there is an abnormality in the breast implant based on a pressure value indicated by the pressure signal received by the sensor signal receiving unit and the first tolerance and the second tolerance stored in the storing unit. The sensor signal receiving unit is configured to receive a first pressure signal from the sensor module in a predetermined period or in an arbitrary manner by an operation of a user. The abnormality determining unit is configured to compare a first variance indicating a difference between a current first pressure signal and a previous first pressure signal with the first tolerance. When the first variance is larger than the first tolerance, the sensor signal receiving unit is configured to receive a second pressure signal from the sensor module after a predetermined first time lapse. The abnormality determining unit is configured to compare a second variance indicating a difference between the first pressure signal and the second pressure signal with the first tolerance, when the second variance is larger than the first tolerance, to compare an absolute value of a difference between the first variance and the second variance with the second tolerance, and when the absolute value of the difference between the first variance and the second variance is smaller than the second tolerance, to determine the abnormality in the breast implant.

According to some embodiments of the present disclosure, a system for sensing abnormality of a breast implant includes a user terminal configured to receive a pressure signal indicating a pressure applied to a breast implant inserted in a body from at least one sensor module disposed on the breast implant and to transmit the pressure signal to outside and a remote server configured to receive the pressure signal from the user terminal and to determine whether there is an abnormality in the breast implant based on the pressure signal. The user terminal includes a sensor signal receiving unit configured to receive the pressure signal indicating the pressure applied to the breast implant from the sensor module and a signal transmitting unit configured to transmit the pressure signal received by the sensor signal receiving unit to the remote server. The remote server includes a signal receiving unit configured to receive the pressure signal from the user terminal, a storing unit configured to store a predetermined reference pressure value, a predetermined first tolerance, and a predetermined second tolerance, and an abnormality determining unit configured to determine whether there is an abnormality in the breast implant based on a pressure value indicated by the pressure signal received by the sensor signal receiving unit and the reference pressure value, the first tolerance, and the second tolerance stored in the storing unit. The signal receiving unit is configured to receive a first pressure signal from the sensor module in a predetermined period or in an arbitrary manner by an operation of a user. The abnormality determining unit is configured to compare a first pressure value indicated by the first pressure signal with the reference pressure value. When an absolute value of a difference between the first pressure value and the reference pressure value is larger than the first tolerance, the sensor signal receiving unit is configured to receive a second pressure signal from the sensor module after a predetermined first time lapse. The abnormality determining unit is configured to compare a second pressure value indicated by the second pressure signal with the reference pressure value, when an absolute value of a difference between the second pressure value and the reference pressure value is larger than the first tolerance, to compare an absolute value of a difference between the first pressure value and the second pressure value with the second tolerance, and when the absolute value of the difference between the first pressure value and the second pressure value is smaller than the second tolerance, to determine the abnormality in the breast implant.

According to some embodiments of the present disclosure, a system for sensing abnormality of a breast implant includes a user terminal configured to receive a pressure signal indicating a pressure applied to a breast implant inserted in a body from at least one sensor module disposed on the breast implant and to transmit the pressure signal to outside and a remote server configured to receive the pressure signal from the user terminal and to determine whether there is an abnormality in the breast implant based on the pressure signal. The user terminal includes a sensor signal receiving unit configured to receive the pressure signal indicating the pressure applied to the breast implant from the sensor module and a signal transmitting unit configured to transmit the pressure signal received by the sensor signal receiving unit to the remote server. The remote server includes a signal receiving unit configured to receive the pressure signal from the user terminal, a storing unit configured to store a predetermined first tolerance and a predetermined second tolerance, and an abnormality determining unit configured to determine whether there is an abnormality in the breast implant based on a pressure value indicated by the pressure signal received by the sensor signal receiving unit and the first tolerance and the second tolerance stored in the storing unit. The signal receiving unit is configured to receive a first pressure signal from the sensor module in a predetermined period or in an arbitrary manner by an operation of a user. The abnormality determining unit is configured to compare a first variance indicating a difference between a current first pressure signal and a previous first pressure signal with the first tolerance. When the first variance is larger than the first tolerance, the signal receiving unit is configured to receive a second pressure signal from the sensor module after a predetermined first time lapse. The abnormality determining unit is configured to compare a second variance indicating a difference between the first pressure signal and the second pressure signal with the first tolerance, when the second variance is larger than the first tolerance, to compare an absolute value of a difference between the first variance and the second variance with the second tolerance, and the absolute value of the difference between the first variance and the second variance is smaller than the second tolerance, to determine the abnormality in the breast implant.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a schematic diagram of a system and an apparatus for sensing an abnormality of a breast implant according to some embodiments of the present disclosure;

FIG. 2 is a functional block diagram of an apparatus for sensing an abnormality of a breast implant according to some embodiments of the present disclosure;

FIGS. 3 and 4 are flowcharts for explaining a method of sensing an abnormality of a breast implant according to some embodiments of the present disclosure;

FIG. 5 is a functional block diagram an apparatus for sensing an abnormality of a breast implant in a system for sensing an abnormality of a breast implant according to some embodiments of the present disclosure;

FIG. 6 is a functional block diagram a server for sensing an abnormality of a breast implant in a system for sensing an abnormality of a breast implant according to some embodiments of the present disclosure;

FIG. 7 is a schematic diagram of a breast implant including a sensor patch according to some embodiments of the present disclosure;

FIGS. 8A and 8B are schematic diagrams patches each including a pressure sensor according to some embodiments of the present disclosure;

FIG. 9 is an exploded view of a sensor module according to some embodiments of the present disclosure;

FIG. 10A is a schematic diagram for explaining pressures applied to a breast implant inserted in a breast in some embodiments of the present disclosure; and

FIG. 10B is a schematic diagram showing an example of attaching a patch including a sensor module according to some embodiments of the present.

### DETAILED DESCRIPTION

Such breast implants are generally manufactured using a cohesive gel that is a silicone having a high cohesive force. The cohesive gel has an advantage in that an outer cover (shell) is strong and a shape is naturally formed after a surgery, remedying disadvantages of a conventional silicone gel (see, for example, Korean Pat. No. 10-1235284, Korean Pat. No. 10-1966979, and Japanese Pat. Laid-Open No. 2010-534551).

Popular side effects seen after a surgery for reconstructing a breast with a breast implant include rupture and capsular contracture.

When a rupture is generated and a silicone component is remained in the body for a long time, the silicone component may permeate through the breast tissue, possibly causing a breast feeding hard and a difficulty in a breast cancer examination. Sometimes the silicone penetrates into a lymph node due to the rupture of the breast implant, and in this case, the penetrated silicone may be incompletely removed and a lymphedema after removing the silicone or a spreading of the silicone to the whole body may occur.

The capsular contracture is a phenomenon generated from thickening of a capsule newly formed surrounding a breast implant, which is generated within the first two years after a surgery, which is one of the most popular side effects of the breast reconstruction employing the breast implant.

A capsular contracture causes deformation and pain, and in a worse case, a reoperation is needed to remove the breast implant and to remove the capsule.

In order to examine the two representative side effects after inserting the breast implant, a patient needs to visit a hospital to consult a doctor and to carry out an image medical inspection such as an ultrasound or an MRI.

Even without a particular symptom, it is recommended to carry out an ultrasound examination every year after inserting the breast implant.

In order to sense abnormalities in such breast implants, for example, US Pat. Pub. No 2009/0012372 describes a system and a method for sensing for the rupture of an implant.

However, when the abnormality of the implant is determined simply by attaching a sensor on the implant and receiving an abnormality signal from the sensor, there may be a risk of a false positive or a false negative.

Particularly, although it is problematic that no abnormality is determined even with an actual abnormality in the case of, for example, a breast implant, it would be worse if an abnormality is determined without an actual abnormality because an operation may be needed to remove a normal breast implant and to insert a new breast implant.

Exemplary embodiments of the present disclosure are described in detail below with reference to the accompanying drawings.

It is an object of the present disclosure to provide an apparatus for sensing an abnormality of a breast implant, which can minimize a false positive and a false negative by sensing the abnormality of the breast implant in an effective manner after a surgery.

It is another object of the present disclosure to provide a system for sensing an abnormality of a breast implant, which can minimize a false positive and a false negative by sensing the abnormality of the breast implant in an effective manner after a surgery.

The challenges to be addressed by the present disclosure are not limited to those mentioned above, and other unmentioned problems can be clearly understood by those skilled in the art from the following description.

A breast implant according to some embodiments of the present disclosure is manufactured with, for example, a silicone material and is used for a plastic surgery of a breast, a hip, or the like, which is inserted in a body and forms and maintains a consistent shape in the body.

Although a breast implant for reconstructing a breast is explained as an example in the present specification, the breast implant and the apparatus for sensing an abnormality of a breast implant according to some embodiments of the present disclosure can be applied to most implants including a shell and a gel-type filler and being inserted in a body for a specific purpose.

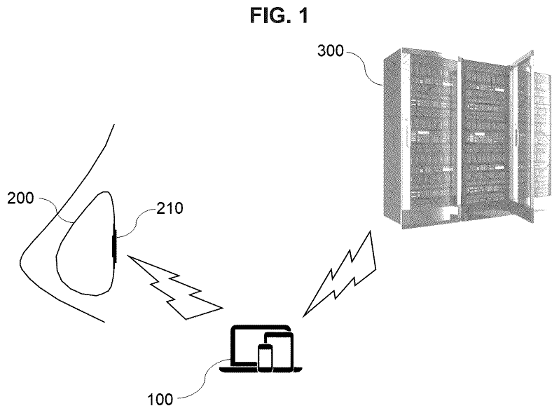

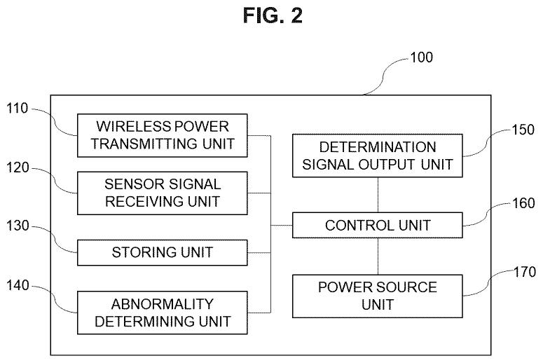

FIG. 1 is a schematic diagram of a system and an apparatus for sensing an abnormality of a breast implant according to some embodiments of the present disclosure. FIG. 2 is a functional block diagram of the apparatus for sensing an abnormality of a breast implant according to some embodiments of the present disclosure.

As shown in FIG. 1, the system for sensing an abnormality of a breast implant according to some embodiments of the present disclosure includes an apparatus 100 for sensing an abnormality of a breast implant 200 configured to receive a pressure signal indicating a pressure applied to the breast implant 200 from at least one sensor module 210 disposed on the breast implant 200 inserted in a body (breast) and to sense an abnormality of the breast implant 200 based on the pressure signal received from the sensor module 210 and a server 300 that is remotely connected with the apparatus 100 and configured to receive various pieces of data from the apparatus 100 and to store received data.

In some embodiments of the present disclosure, the apparatus 100 is configured to receive a pressure signal indicating a pressure applied to the breast implant 200 from the sensor module 210 disposed on the breast implant 200, to output a determination signal by determining an abnormality in the breast implant 200 based on the pressure signal received from the sensor module 210, and to transmit various pieces of data including the pressure signal and the determination signal to the server 300 to allow the data to be stored in the server 300.

As shown in FIG. 2, the apparatus 100 according to some embodiments of the present disclosure includes a wireless power transmitting unit 110 configured to wirelessly transmit a power to the sensor module 210 disposed on the breast implant 200 that is to be inserted in a body (breast), a sensor signal receiving unit 120 configured to receive the pressure signal indicating the pressure applied to the breast implant 200 from the sensor module 210, a storing unit 130 configured to store a predetermined reference pressure value and a predetermined first tolerance, an abnormality determining unit 140 configured to determine an abnormality of the breast implant 200 based on the pressure signal received by the sensor signal receiving unit 120 and the reference pressure value and the first tolerance stored in the storing unit 130, a determination signal output unit 150 configured to output a determination signal indicating an abnormality or no abnormality in the breast implant 200 determined by the abnormality determining unit 140 based on the pressure signal, the reference pressure value, and the first tolerance, a control unit 160 configured to control the units 110 to 150, and a power source unit 170 configured to supply a power to the units 110 to 150 through the control unit 160.

In some embodiments of the present disclosure, the control unit 160 controls the sensor signal receiving unit 120 to receive a first pressure signal from the sensor module 210 in a predetermined (set) period or in an arbitrary manner by an operation of a user, the abnormality determining unit 140 to compare a first pressure value (P1) indicated by the first pressure signal and the reference pressure value (PR), when an absolute value of a difference between the first pressure value (P1) and the reference pressure value (PR) is larger than the first tolerance (T1), the sensor signal receiving unit 120 to receive a second pressure signal from the sensor module 210 after a predetermined first time lapse, the abnormality determining unit 140 to compare a second pressure value (P2) indicated by the second pressure signal with the reference pressure value (PR), and when an absolute value of a difference between the second pressure value (P2) and the reference pressure value (PR) is larger than the first tolerance (T1), the determination signal output unit 150 to output a determination signal indicating an abnormality in the breast implant 200.

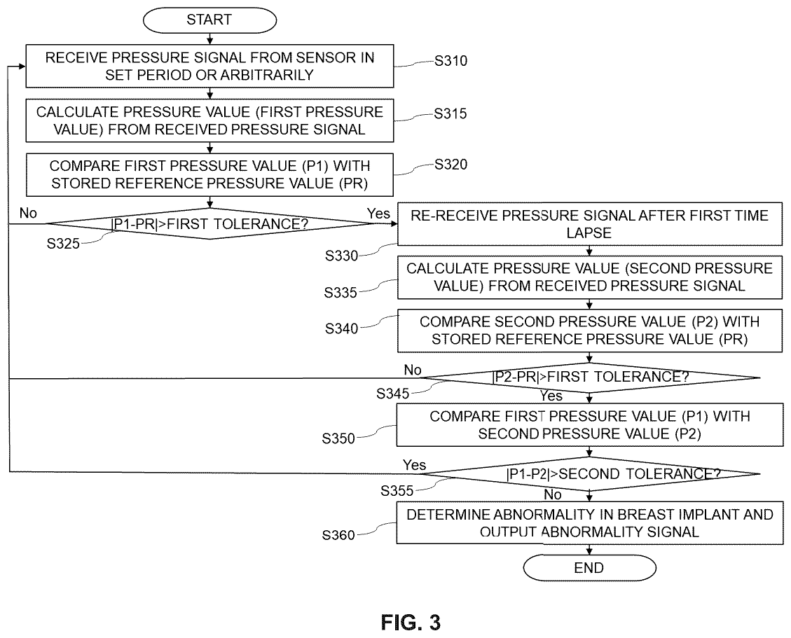

FIG. 3 is a flowchart for explaining a method of sensing an abnormality of the breast implant 200 according to some embodiments of the present disclosure.

In the following descriptions, each operation is performed by the control unit 160 to control each corresponding function module implemented as a program module in a processor and each corresponding hardware component.

In step S310, a pressure signal is received from a sensor (sensor module) of a breast implant in a predetermined period or by an arbitrary operation of a user.

Thereafter, a pressure value (first pressure value (P1)) applied to the breast implant is calculated from the pressure signal received in step S310 (step S315). The first pressure value (P1) calculated in the above manner is then compared with a reference pressure value (PR) stored in advance (step S320).

In some embodiments of the present disclosure, the reference pressure value (PR) is an actual pressure value measured in a predetermined stabilization time after a surgery and stored in a storing unit (memory). In some embodiments of the present disclosure, the reference pressure value (PR) is updated by re-measuring the actual pressure value in a predetermined time period.

That is, the reference pressure value (PR) indicates a pressure applied to the breast implant in a normal state; and therefore, when a pressure value indicated by a received pressure signal is larger or smaller than the reference pressure value (PR) by a predetermined amount, it is possible to determine that a rupture or a capsular contracture has occurred in the breast implant.

As a result of comparing the calculated first pressure value (P1) with the stored reference pressure value (PR), if an absolute value of a difference between the first pressure value (P1) and the reference pressure value (PR) is larger than the stored first tolerance (T1) (step S325, Yes), a second pressure signal is received from the sensor module after a predetermined first time lapse (step S330).

A pressure value (second pressure value (P2)) is calculated from the received second pressure signal (step S335). The calculated second pressure value (P2) is then compared with the stored reference pressure value (PR) (step S340).

As a result of comparing the second pressure value (P2) with the reference pressure value (PR), if an absolute value of a difference between the second pressure value (P2) and the reference pressure value (PR) is larger than the first tolerance (T1) (step S345, Yes), a determination signal indicating an abnormality in the breast implant is outputted (step S360).

As large and small pressures can be constantly applied to a breast implant inserted in a body, if an abnormality is determined with a single measurement, there always exists a risk of a false positive or a false negative.

For example, as a result of comparing the first pressure value (P1) with the reference pressure value in step S325, if the absolute value of the difference between the first pressure value (P1) and the reference pressure value is larger than the first tolerance (T1), that is, if it is determined that an abnormal pressure is applied to the sensor disposed on the breast implant, it may be necessary to determine whether such an abnormal pressure is temporary or persisting.

To this end, in some embodiments of the present disclosure, once it is determined that the absolute value of the difference between the first pressure value (P1) and the reference pressure value (PR) is larger than the first tolerance (T1), the second pressure signal is received from the sensor module after the predetermined first time lapse.

In some embodiments of the present disclosure, upon receiving the pressure signal from the sensor module in the predetermined period, when the first pressure value (P1) is larger than the reference pressure value (PR), the control unit receives the second pressure signal from the sensor module after the first time lapse, ignoring the predetermined period.

In some embodiments of the present disclosure, upon receiving the pressure signal from the sensor module by an arbitrary operation of a user, when the first pressure value (P1) is larger than the reference pressure value (PR), the control unit receives the second pressure signal from the sensor module after the first time lapse, ignoring the operation of the user.

The first time is a time for determining whether the abnormal pressure is applied to the sensor disposed on the breast implant temporarily or constantly, which can be set equal to or longer or shorter than the predetermined period.

In order to further reduce the risk of the false positive and the false negative, in some embodiments of the present disclosure, as a result of comparing the second pressure value (P2) with the reference pressure value (PR), if the absolute value of the difference between the second pressure value (P2) and the reference pressure value (PR) is larger than the first tolerance (T1) (step S345, Yes), the first pressure value (P1) is compared with the second pressure value (P2) (step S350).

As a result of comparing the first pressure value (P1) with the second pressure value (P2), if an absolute value of a difference between the first pressure value (P1) and the second pressure value (P2) is smaller than a second tolerance (T2) (step S355, No), a determination signal indicating an abnormality in the breast implant is outputted (step S360).

The first pressure value (P1) is compared with the second pressure value (P2) in the above manner because, if there is a big difference between the pressure values before and after the first time, it is possible to determined that the abnormal pressure is more likely applied to the breast implant in a temporal manner, and if there is a small difference between the pressure values before and after the first time within the second tolerance, it is possible to determine that the abnormal pressure is applied to the breast implant constantly for the first time, meaning that an abnormality has occurred in the breast implant.

In this manner, the apparatus 100 according to some embodiments of the present disclosure can sense an abnormality in a breast implant after a surgery in an effective manner, thus minimizing the risk of the false positive and the false negative.

FIG. 4 is a flowchart for explaining a method of sensing an abnormality of the breast implant 200 according to some embodiments of the present disclosure.

In step S410, a pressure signal (first pressure signal) is received from a sensor (sensor module) of a breast implant in a predetermined period or by an arbitrary operation of a user.

After receiving a series of first pressure signals including at least two first pressure signals, a first variation (V1) indicating a difference between a current first pressure signal and a previous first pressure signal is calculated (step S415). The calculated first variance (V1) is then compared with a first tolerance (T1) stored in advance (step S420).

As a result of comparing the first variance (V1) with the first tolerance (T1), if the first variance (V1) is larger than the first tolerance (T1) (step S425, Yes), a second pressure signal is received from the sensor module after a predetermined first time lapse (step S430).

A second variance (V2) indicating a difference between the first pressure signal and the second pressure signal is calculated (step S435). The calculated second variance (V2) is then compared with the first tolerance (T1) (step S440).

As a result of comparing the second variance (V2) with the first tolerance (T1), if the second variance (V2) is larger than the first tolerance (T1) (step S445, Yes), a determination signal indicating an abnormality in the breast implant is outputted (step S460).

As large and small pressures can be constantly applied to a breast implant inserted in a body, if an abnormality is determined with a single measurement, there always exists a risk of a false positive or a false negative.

For example, as a result of comparing the first variance (V1) calculated in step S425 with the first tolerance (T1), if the first variance (V1) is larger than the first tolerance (T1), that is, if it is determined that an abnormal pressure is applied to the sensor disposed on the breast implant, it may be necessary to determine whether such an abnormal pressure is temporary or persisting.

To this end, in some embodiments of the present disclosure, once it is determined that the first variance (V1) is larger than the first the second pressure signal is received from the sensor module after the predetermined first time lapse.

In some embodiments of the present disclosure, upon receiving the pressure signal from the sensor module in the predetermined period, when the first variance (V1) is larger than the first tolerance (T1), the control unit receives the second pressure signal from the sensor module after the first time lapse, ignoring the predetermined period.

In some embodiments of the present disclosure, upon receiving the pressure signal from the sensor module by an arbitrary operation of a user, when the first variance (V1) is larger than the first tolerance (T1), the control unit receives the second pressure signal from the sensor module after the first time lapse, ignoring the operation of the user.

The first time is a time for determining whether the abnormal pressure is applied to the sensor disposed on the breast implant temporarily or constantly, which can be set equal to or longer or shorter than the predetermined period.

In order to further reduce the risk of the false positive and the false negative, in some embodiments of the present disclosure, as a result of comparing the second variance (V2) with the first tolerance (T1), if the second variance (V2) is larger than the first tolerance (T1) (step S445, Yes), the first variance (V1) is compared with the second variance (V2) (step S450).

As a result of comparing the first variance (V1) with the second variance (V2), if an absolute value of a difference between the first variance (V1) and the second variance (V2) is smaller than a second tolerance (T2) (step S455, No), a determination signal indicating an abnormality in the breast implant is outputted (step S460).

The first variance (V1) is compared with the second variance (V2) in the above manner because, if there is a big difference between the pressure values before and after the first time, it is possible to determined that the abnormal pressure is more likely applied to the breast implant in a temporal manner, and if there is a small difference between the pressure values before and after the first time within the second tolerance, it is possible to determine that the abnormal pressure is applied to the breast implant constantly for the first time, meaning that an abnormality has occurred in the breast implant.

In this manner, the apparatus 100 according to some embodiments of the present disclosure can sense an abnormality in a breast implant after a surgery in an effective manner, thus minimizing the risk of the false positive and the false negative.

In some embodiments of the present disclosure, the sensor signal receiving unit 120 further receives a bio-signal indicating at least one of heart rate, pulse, body temperature, blood pressure, breathing rate, and blood glucose from the sensor module 210, and the storing unit 130 further stores the bio-signal received by the sensor signal receiving unit 120.

In some embodiments of the present disclosure, the control unit 160 stores various pieces of data including the pressure signal, the determination signal, and the bio-signal in the storing unit 130, and in some embodiments of the present disclosure, the control unit 160 transmits the data to the server 300 so that the server 300 stores therein the data.

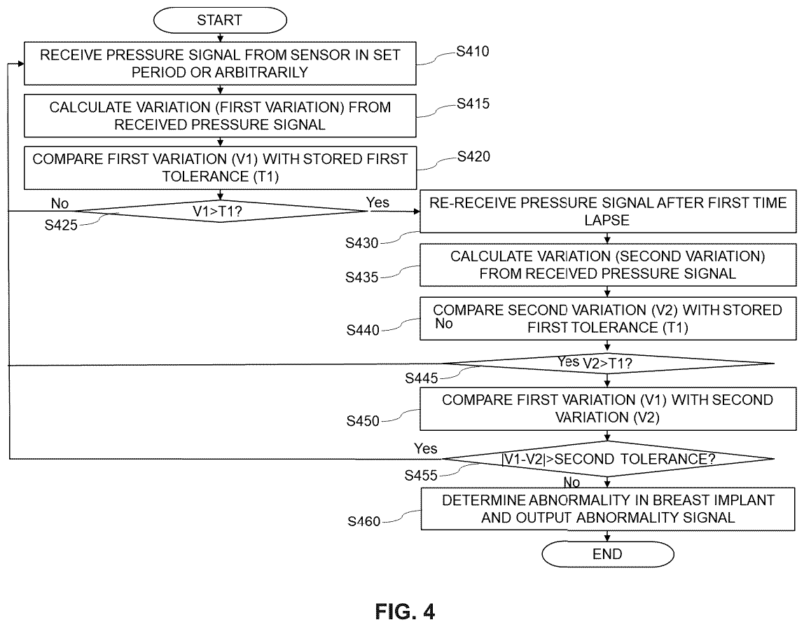

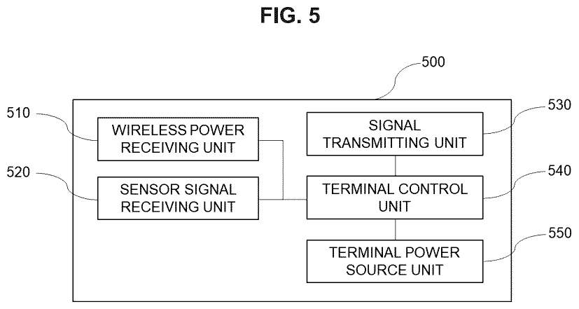

FIG. 5 is a functional block diagram an apparatus for sensing an abnormality of a breast implant in a system for sensing an abnormality of a breast implant according to some embodiments of the present disclosure. FIG. 6 is a functional block diagram a server for sensing an abnormality of a breast implant in a system for sensing an abnormality of a breast implant according to some embodiments of the present disclosure.

The system for sensing an abnormality of a breast implant according to some embodiments of the present disclosure includes an apparatus (user terminal) 500 for sensing an abnormality in a breast implant configured to receive a pressure signal indicating a pressure applied to a breast implant from at least one sensor module dispose on the breast implant inserted in a body and to transmit the pressure signal to outside and a server (remote server) 600 for sensing an abnormality of a breast implant configured to receive the pressure signal from the user terminal 500 and to determine an abnormality in the breast implant based on the received pressure signal.

In some embodiments of the present disclosure, the user terminal 500 includes a wireless power transmitting unit 510 configured to wirelessly transmit a power to the sensor module 210, a sensor signal receiving unit 520 configured to receive a pressure signal indicating a pressure applied to the breast implant 200 from the sensor module 210, a signal transmitting unit 530 configured to transmit the pressure signal received by the sensor signal receiving unit 520 to the remote server 600, a terminal control unit 540 configured to control the units 510 to 530, and a terminal power source unit 550 configured to supply a power to the units 510 to 530 through the terminal control unit 540.

In some embodiments of the present disclosure, the remote server 600 includes a signal receiving unit 610 from the user terminal 500, a storing unit 620 configured to store a predetermined reference pressure value and a predetermined first tolerance, an abnormality determining unit 630 configured to determine an abnormality of the breast implant based on the pressure signal received from the user terminal 500 and the reference pressure value and the first tolerance stored in the storing unit 620, a determination signal output unit 640 configured to output a determination signal indicating an abnormality or no abnormality in the breast implant determined by the abnormality determining unit 630 based on the pressure signal, the reference pressure value, and the first tolerance, a server control unit 650 configured to control the units 610 to 640, and a server power source unit 660 configured to supply a power to the units 610 to 640 through the server control unit 650.

In some embodiments of the present disclosure, the server control unit 650 controls the signal receiving unit 610 to receive a first pressure signal from the user terminal 500, which is received from the sensor module 210 in a predetermined (set) period or in an arbitrary manner by an operation of a user, the abnormality determining unit 630 to compare a first pressure value (P1) indicated by the first pressure signal and the reference pressure value (PR), when an absolute value of a difference between the first pressure value (P1) and the reference pressure value (PR) is larger than the first tolerance (T1), the signal receiving unit 610 to receive a second pressure signal from the user terminal 500, which is received from the sensor module 210 after a predetermined first time lapse, the abnormality determining unit 630 to compare a second pressure value (P2) indicated by the second pressure signal with the reference pressure value (PR), and when an absolute value of a difference between the second pressure value (P2) and the reference pressure value (PR) is larger than the first tolerance (T1), the determination signal output unit 640 to output a determination signal indicating an abnormality in the breast implant 200.

In some embodiments of the present disclosure, the signal receiving unit 610 is configured to receive the pressure signal from the user terminal 500, the storing unit 620 is configured to store a predetermined first tolerance, the abnormality determining unit 630 is configured to determine an abnormality in the breast implant 200 based on the pressure signal received from the user terminal 500 and the first tolerance stored in the storing unit 620, the determination signal output unit 640 is configured to output a determination signal indicating an abnormality in the breast implant 200 determined by the abnormality determining unit 630 based on the pressure signal and the first tolerance, the server control unit 650 is configured to control the units 610 to 640, and the server power source unit 660 is configured to supply a power to the units 610 to 640 through the server control unit 650.

In some embodiments of the present disclosure, the server control unit 650 controls the signal receiving unit 610 to receive a first pressure signal from the user terminal 500, which is received from the sensor module 210 in a predetermined (set) period or in an arbitrary manner by an operation of a user, the abnormality determining unit 630 to compare a first variance (V1) indicating a difference between the current first pressure signal and the previous first pressure signal with the first tolerance (T1), when the first variance (V1) is larger than the first tolerance (T1), the signal receiving unit 610 to receive a second pressure signal from the user terminal 500, which is received from the sensor module 210 after a predetermined first time lapse, the abnormality determining unit 630 to compare a second variance (V2) indicating a difference between the first pressure signal and the second pressure signal, and if the second variance (V2) is larger than the first tolerance (T1), the determination signal output unit 640 to output a determination signal indicating an abnormality in the breast implant 200.

In some embodiments of the present disclosure, the sensor signal receiving unit 520 further receives a bio-signal indicating at least one of heart rate, pulse, body temperature, blood pressure, breathing rate, and blood glucose from the sensor module 210, and the signal transmitting unit 530 transmits the bio-signal received by the sensor signal receiving unit 520 alone or with the pressure signal and the determination signal to outside.

In this manner, the user terminal 500 shown in FIG. 5 is configured to receive the pressure signal from the sensor module 210 and to transmit the pressure signal to the remote server 600 without storing the pressure signal or determining an abnormality of the breast implant, which makes the structure simple.

The pressure signal transmitted from the user terminal 500 is received by the remote server 600 and stored in the storing unit 620 of the remote server 600 so that the abnormality of the breast implant is determined in the remote server 600. When the bio-signal is transmitted from the user terminal 500, the remote server 600 receives the bio-signal and stores the bio-signal in the storing unit 620.

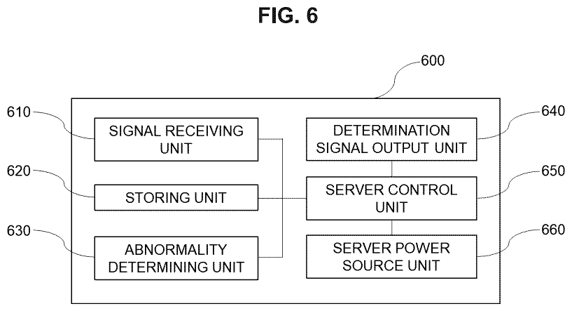

FIG. 7 is a schematic diagram of the breast implant 200 including a sensor patch according to some embodiments of the present disclosure.

The breast implant 200 according to some embodiments of the present disclosure is manufactured with, for example, a silicone material and is used for a plastic surgery of a breast, a hip, or the like, which is inserted in a body and forms and maintains a consistent shape in the body.

Although a breast implant for reconstructing a breast is explained as an example in the present specification, the breast implant and the apparatus for sensing an abnormality of a breast implant according to some embodiments of the present disclosure can be applied to most implants including a shell and a gel-type filler and being inserted in a body for a specific purpose.

As shown in FIG. 7, the breast implant 200 according to some embodiments of the present disclosure includes a shell 201 forming an outer cover of teardrop type or round type, a silicone gel as a filler injected into the shell 201, and a patch 202 for sealing an injection hole for injecting the silicone gel.

The patch 202 according to some embodiments of the present disclosure includes a sensor module 210 including a pressure sensor for sensing a pressure applied to the breast implant 200.

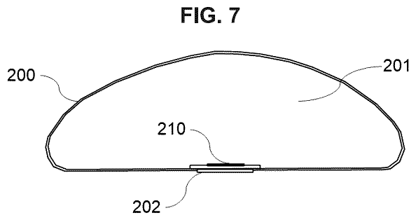

FIGS. 8A and 8B are schematic diagrams patches each including a pressure sensor according to some embodiments of the present disclosure.

The patch 202 includes a first barrier (inner barrier) 202-1 forming a first side facing inside the breast implant 200 upon being attached on the breast implant 200 with the filler of the gel state injected, a second barrier (outer barrier) 202-2 facing the first barrier 202-1 and forming a second side opposite to the first side. The sensor module 210 is disposed on the first side of the first barrier 202-1.

In the patch 202 shown in FIG. 8B, the sensor module 210 is disposed between the first barrier 202-1 and the second barrier 202-2.

Although the first barrier 202-1 and the second barrier 202-2 are shown separately in FIGS. 8A and 8B for the sake of explanation, the patch 202 is a unit member formed by a first barrier 211 and a second barrier 212 making contact with each other. The first barrier 202-1 forms the inner side of the patch 202, and the second barrier 202-2 forms the outer side of the patch 202.

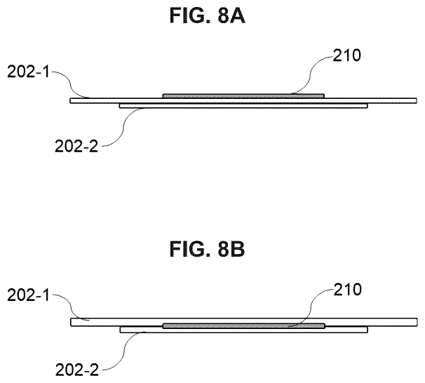

FIG. 9 is an exploded view of the sensor module 210 according to some embodiments of the present disclosure.

As shown in FIG. 9, the sensor module 210 is formed as a film including two protective films 910, 930 and a substrate 920 between the two protective films 910, 930. On the substrate 920, at least one pressure sensor 923 for sensing a pressure applied on the breast implant and outputting a pressure signal, a controller 922 for transmitting the pressure signal from the pressure sensor 923 to the outside, and a wireless power receiving unit 921 for receiving a power from the outside and providing the power to the controller 922.

The pressure sensor 923 is an element that, when a predetermined pressure or force is applied, generates a signal proportional to the pressure or the force applied. The pressure sensor 923 includes a diaphragm sensor that curves according to the pressure or the force applied, a force sensing resistor (FSR) including two conductive plate and a conductive polymer between the two conductive plate, and a piezoelectric element that generates a voltage when a pressure is applied.

A tension sensor that senses a tension of the shell of the breast implant can be used instead of the pressure sensor; however, as a change of the tension is also a physical amount proportional to a change of the pressure due to the rupture or the capsular contracture of the breast implant, the tension sensor is considered as a type of the pressure sensor in the present specification.

In some embodiments of the present disclosure, the sensor module 210 further includes a battery 924 for storing the power received by the wireless power receiving unit 921 in a wireless manner.

That is, the sensor module 210 according to some embodiments of the present disclosure can operate either by receiving the power in a wireless manner without a battery or by charging a battery with the power received in a wireless manner.

In some embodiments of the present disclosure, the sensor module 210 includes at least one of a heart rate sensor, a pulse sensor, a body temperature sensor, a blood pressure sensor, a breathing rate sensor, and a blood glucose sensor.

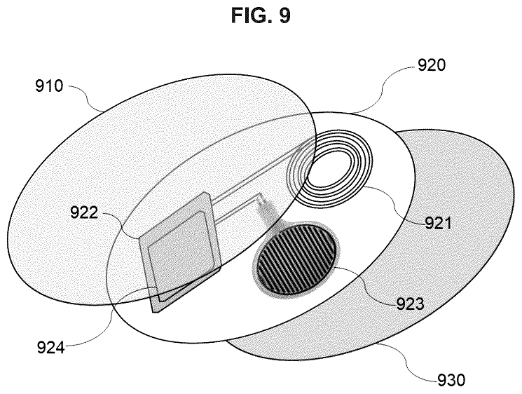

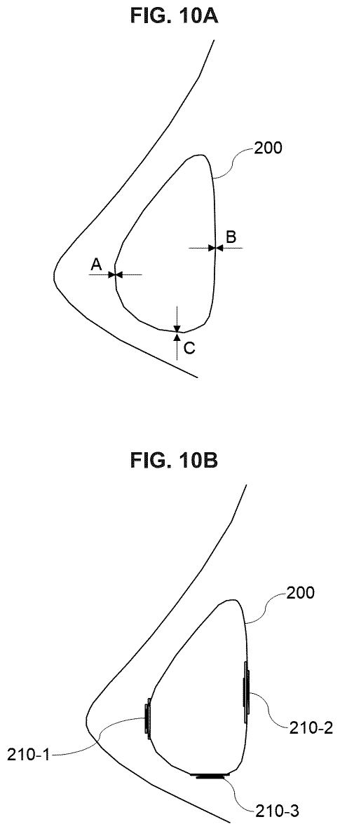

FIG. 10A is a schematic diagram for explaining pressures applied to the breast implant 200 inserted in a breast in some embodiments of the present disclosure. FIG. 10B is a schematic diagram showing an example of attaching a patch including a sensor module according to some embodiments of the present.

As shown in FIG. 10A, when the breast implant 200 is inserted in a breast, in a normal state in which no external pressure is applied on the breast, a pressure A is applied on the breast implant 200 from outside a body, a pressure B is applied on the breast implant 200 from or against inside the body, and pressure C is applied on the breast implant 200 due to the gravity.

Typically, a silicone gel is inserted in a shell of a breast implant. The breast implant is sealed by a patch at a position on the bottom of the breast implant (near a position indicated by B) in a manner that a consistent tension is maintained. However, when a part of the breast implant is ruptured or a capsular contracture is generated at a part of the breast implant, at least one of the pressures A, B, and C is changed.

In the case of a cohesive-gel implant, which is widely used in these days, even if a part of the breast implant is ruptured after inserting the breast implant in the breast, a patient can hardly feel an abnormality, which is referred to as a silent rupture. That is, the cohesive gel keeps the gel state even when the breast implant is ruptured.

An image inspection by a magnetic resonance imaging (MRI) is so far one of the most precise methods to inspect a rupture of the breast implant. However, the MRI itself still shows a false positive and a false negative and can be a burden to a patient in terms of the cost.

When a foreign material comes into a body, a capsule is formed around the foreign material. Such capsule excessively formed and hardened causes a capsular contracture.

When a breast implant is inserted in a breast, the blood platelet in the blood is activated and a material called the transforming growth factor β (TGF-β) is secreted. The TGF-β takes a role of gathering monocytes around the breast implant where an inflammation is generated. The monocyte is a sort of the white blood cell, which develops into a macrophage near the inflammation and secretes the TGF-β again. This results in a fibroblast near the inflammation and a collagen synthesized from the fibroblast generates the capsular contracture.

As shown in FIG. 10A, when a breast implant is inserted in a breast, in a normal state in which no external pressure is applied on the breast, a pressure A is applied on the breast implant 200 from outside a body, a pressure B is applied on the breast implant 200 from or against inside the body, and pressure C is applied on the breast implant 200 due to the gravity.

Therefore, with a patch including a pressure sensor disposed on at least one of sites A, B, and C, it is possible to notify a user of an abnormality of the breast implant by sending a change of the pressure when the abnormality occurred on the breast implant.

As shown in FIG. 10B, a patch including a sensor module 210-1 can be disposed on the site A, a patch including a sensor module 210-2 can be disposed on the site B, and a patch including a sensor module 210-3 can be disposed on the site C.

In some embodiments of the present disclosure, the apparatus for sensing the abnormality of the breast implant and the system for sensing the abnormality of the breast implant store the reference pressure and the first tolerance for each of a plurality of positions, for example, the sites A, B, and C shown in FIG. 10A, in the memory.

The apparatus for sensing the abnormality of the breast implant and the system for sensing the abnormality of the breast implant according to some embodiments of the present disclosure are supposed to output an abnormality of the breast implant when, for example, a difference between at least one of a plurality of pressure signals received respectively from the sensor modules disposed at a plurality of sites and a reference pressure stored in association with the corresponding site exceeds a predetermined value.

The apparatus for sensing the abnormality of the breast implant and the system for sensing the abnormality of the breast implant according to some embodiments of the present disclosure can set the first tolerance for determining the abnormality in a pressure signal received from one sensor module larger than the first tolerances for determining the abnormality in pressure signals received from the other sensor modules.

With this configuration, by outputting the abnormality upon sensing an abnormality in one site when the magnitude of the abnormality is relatively large and not outputting the abnormality when the magnitude of the abnormality is relatively small and when the abnormality is in an acceptable range, it is possible to prevent from unnecessarily outputting the abnormality.

The apparatus for sensing the abnormality of the breast implant and the system for sensing the abnormality of the breast implant according to some embodiments of the present disclosure can set different first tolerances for a plurality of sites from each other to determine the abnormality.

The apparatus for sensing the abnormality of the breast implant and the system for sensing the abnormality of the breast implant according to some embodiments of the present disclosure can set, for example, the first tolerance for the bottom of the breast implant (the site C in FIG. 10A) larger or smaller than those for the other sites.

With this configuration, it is possible to make a proper determination considering the change in the pressure depending on the site of the breast implant.

According to some embodiments of the present disclosure, when there is a change in the pressure due to the rupture or the capsular contracture, a user is notified of the rupture or the capsular contracture of the breast implant according to the change of the pressure, to enable the user to take a proper action at the right time.

This can help the user to remove an anxiety about maintaining the breast with safety for a long time after inserting the breast implant in the breast, and at the same time, to relieve an inconvenience of taking an ultrasound inspection or an MRI inspection on a regular basis.

As described above, according to some embodiments of the present disclosure, it is possible to provide an apparatus for sensing an abnormality of a breast implant, which can minimize a false positive and a false negative by sensing the abnormality of the breast implant in an effective manner after a surgery.

Further, according to some embodiments of the present disclosure, it is possible to provide a system for sensing an abnormality of a breast implant, which can minimize a false positive and a false negative by sensing the abnormality of the breast implant in an effective manner after a surgery.

The present disclosure should not be limited to these embodiments but various changes and modifications are made by one ordinarily skilled in the art within the subject matter, the spirit and scope of the present disclosure as hereinafter claimed. Specific terms used in this disclosure and drawings are used for illustrative purposes and not to be considered as limitations of the present disclosure. Exemplary embodiments of the present disclosure have been described for the sake of brevity and clarity. Accordingly, one of ordinary skill would understand the scope of the claimed invention is not to be limited by the explicitly described above embodiments but by the claims and equivalents thereof.

What is claimed is:

1. An apparatus for sensing abnormality of a breast implant, comprising:
a sensor signal receiver configured to receive a pressure signal indicating a pressure applied to a breast implant inserted in a body from at least one sensor module disposed on the breast implant;
a memory configured to store a predetermined reference pressure value, a predetermined first tolerance, and a predetermined second tolerance; and
an abnormality determining processor configured to determine whether there is an abnormality in the breast implant based on a pressure value indicated by the pressure signal received by the sensor signal receiver and the reference pressure value, the first tolerance, and the second tolerance stored in the memory,
the sensor signal receiver configured to receive a first pressure signal from the sensor module in a predetermined period or in an arbitrary manner by an operation of a user,
the abnormality determining processor configured to compare a first pressure value indicated by the first pressure signal with the reference pressure value,
in response to an absolute value of a difference between the first pressure value and the reference pressure value being larger than the first tolerance, the sensor signal receiver configured to receive a second pressure signal from the sensor module after a predetermined first time lapse, and
the abnormality determining processor configured to:
compare a second pressure value indicated by the second pressure signal with the reference pressure value,
in response to an absolute value of a difference between the second pressure value and the reference pressure value being larger than the first tolerance, compare an absolute value of a difference between the first pressure value and the second pressure value with the second tolerance, and
in response to the absolute value of the difference between the first pressure value and the second pressure value being smaller than the second tolerance, determine the abnormality in the breast implant.

2. The apparatus according to claim 1, wherein:
upon receiving the pressure signal from the sensor module in the predetermined period, in response to the first pressure value being larger than the reference pressure value, the sensor signal receiver is configured to receive the second pressure signal from the sensor module after the first time lapse, regardless of the predetermined period, and
upon receiving the pressure signal from the sensor module by an arbitrary operation of a user, in response to the first pressure value being larger than the reference pressure value, the sensor signal receiver is configured to receive the second pressure signal from the sensor module after the first time lapse, regardless of the operation of the user.

3. The apparatus according to claim 1, wherein:
the sensor module includes a plurality of sensor modules, and
the memory is configured to store different first tolerances for the plurality of sensor modules from each other.

4. The apparatus according to claim 1, wherein:
the sensor module includes a plurality of sensor modules, and
the memory is configured to store the first tolerance for one of the plurality of sensor modules differently from those for other sensor modules.

5. The apparatus according to claim 1, wherein:
the sensor signal receiver is configured to further receive a bio-signal indicating at least one of heart rate, pulse, body temperature, blood pressure, breathing rate, or blood glucose from the sensor module, and
the memory is configured to further store the bio-signal received by the sensor signal receiver.

6. An apparatus for sensing abnormality of a breast implant, comprising:
a sensor signal receiver configured to receive a pressure signal indicating a pressure applied to a breast implant inserted in a body from at least one sensor module disposed on the breast implant;
a memory configured to store a predetermined first tolerance and a predetermined second tolerance; and
an abnormality determining processor configured to determine whether there is an abnormality in the breast implant based on a pressure value indicated by the pressure signal received by the sensor signal receiver and the first tolerance and the second tolerance stored in the memory,
the sensor signal receiver configured to receive a first pressure signal from the sensor module in a predetermined period or in an arbitrary manner by an operation of a user,
the abnormality determining processor configured to compare a first variance indicating a difference between a current first pressure signal and a previous first pressure signal with the first tolerance,
in response to the first variance being larger than the first tolerance, the sensor signal receiver configured to receive a second pressure signal from the sensor module after a predetermined first time lapse, and
the abnormality determining processor configured to:
compare a second variance indicating a difference between the first pressure signal and the second pressure signal with the first tolerance,
in response to the second variance is larger than the first tolerance, compare an absolute value of a difference between the first variance and the second variance with the second tolerance, and
in response to the absolute value of the difference between the first variance and the second variance being smaller than the second tolerance, determine the abnormality in the breast implant.

7. The apparatus according to claim 6, wherein:
upon receiving the pressure signal from the sensor module in the predetermined period, in response to the first variance being larger than the first tolerance, the sensor signal receiver configured to receive the second pressure signal from the sensor module after the first time lapse, regardless of the predetermined period, and
upon receiving the pressure signal from the sensor module by an arbitrary operation of a user, in response to the first variance being larger than the first tolerance, the sensor signal receiver configured to receive the second pressure signal from the sensor module after the first time lapse, regardless of the operation of the user.

8. The apparatus according to claim 6, wherein:
the sensor module includes a plurality of sensor modules, and
the memory is configured to store different first tolerances for the plurality of sensor modules from each other.

9. The apparatus according to claim 6, wherein:
the sensor module includes a plurality of sensor modules, and
the memory is configured to store the first tolerance for one of the plurality of sensor modules differently from those for other sensor modules.

10. The apparatus according to claim 6, wherein:
the sensor signal receiver is configured to further receive a bio-signal indicating at least one of heart rate, pulse, body temperature, blood pressure, breathing rate, or blood glucose from the sensor module, and
the memory is configured to further store the bio-signal received by the sensor signal receiver.

11. A system for sensing abnormality of a breast implant, comprising:
a user terminal configured to receive a pressure signal indicating a pressure applied to a breast implant inserted in a body from at least one sensor module disposed on the breast implant and to transmit the pressure signal to outside; and
a remote server configured to receive the pressure signal from the user terminal and to determine whether there is an abnormality in the breast implant based on the pressure signal, wherein:
the user terminal includes:
a sensor signal receiver configured to receive the pressure signal indicating the pressure applied to the breast implant from the sensor module, and
a signal transmitter configured to transmit the pressure signal received by the sensor signal receiver to the remote server,
the remote server includes:
a signal receiver configured to receive the pressure signal from the user terminal,
a memory configured to store a predetermined reference pressure value, a predetermined first tolerance, and a predetermined second tolerance, and
an abnormality determining processor configured to determine whether there is an abnormality in the breast implant based on a pressure value indicated by the pressure signal received by the sensor signal receiver and the reference pressure value, the first tolerance, and the second tolerance stored in the memory,
the signal receiver is configured to receive a first pressure signal from the sensor module in a predetermined period or in an arbitrary manner by an operation of a user,
the abnormality determining processor is configured to compare a first pressure value indicated by the first pressure signal with the reference pressure value,
in response to an absolute value of a difference between the first pressure value and the reference pressure value being larger than the first tolerance, the sensor signal receiver is configured to receive a second pressure signal from the sensor module after a predetermined first time lapse, and
the abnormality determining processor is configured to:
compare a second pressure value indicated by the second pressure signal with the reference pressure value,
in response to an absolute value of a difference between the second pressure value and the reference pressure value being larger than the first tolerance, compare an absolute value of a difference between the first pressure value and the second pressure value with the second tolerance, and
in response to the absolute value of the difference between the first pressure value and the second pressure value being smaller than the second tolerance, determine the abnormality in the breast implant.

12. The system according to claim 11, wherein:
the sensor signal receiver is configured to further receive a bio-signal indicating at least one of heart rate, pulse, body temperature, blood pressure, breathing rate, or blood glucose from the sensor module,
the signal transmitter is configured to further transmit the bio-signal received by the sensor signal receiver to the remote server,
the signal receiver is configured to further receive the bio-signal from the user terminal, and
the memory is configured to further store the bio-signal received from the user terminal.

13. A system for sensing abnormality of a breast implant, comprising:
a user terminal configured to receive a pressure signal indicating a pressure applied to a breast implant inserted in a body from at least one sensor module disposed on the breast implant and to transmit the pressure signal to outside; and
a remote server configured to receive the pressure signal from the user terminal and to determine whether there is an abnormality in the breast implant based on the pressure signal, wherein:
the user terminal includes:
a sensor signal receiver configured to receive the pressure signal indicating the pressure applied to the breast implant from the sensor module, and
a signal transmitter configured to transmit the pressure signal received by the sensor signal receiver to the remote server,
the remote server includes:
a signal receiver configured to receive the pressure signal from the user terminal,
a memory configured to store a predetermined first tolerance and a predetermined second tolerance, and
an abnormality determining processor configured to determine whether there is an abnormality in the breast implant based on a pressure value indicated by the pressure signal received by the sensor signal receiver and the first tolerance and the second tolerance stored in the memory,
the signal receiver is configured to receive a first pressure signal from the sensor module in a predetermined period or in an arbitrary manner by an operation of a user,
the abnormality determining processor is configured to compare a first variance indicating a difference between a current first pressure signal and a previous first pressure signal with the first tolerance,
in response to the first variance being larger than the first tolerance, the signal receiver is configured to receive a second pressure signal from the sensor module after a predetermined first time lapse,
the abnormality determining processor is configured to:
compare a second variance indicating a difference between the first pressure signal and the second pressure signal with the first tolerance,
in response to the second variance being larger than the first tolerance, compare an absolute value of a difference between the first variance and the second variance with the second tolerance, and
in response to the absolute value of the difference between the first variance and the second variance being smaller than the second tolerance, determine the abnormality in the breast implant.

14. The system according to claim 13, wherein:
the sensor signal receiver is configured to further receive a bio-signal indicating at least one of heart rate, pulse, body temperature, blood pressure, breathing rate, or blood glucose from the sensor module,
the signal transmitter is configured to further transmit the bio-signal received by the sensor signal receiver to the remote server,
the signal receiver is configured to further receive the bio-signal from the user terminal, and
the memory is configured to further store the bio-signal received from the user terminal.
